(19) United States
(12) Patent Application Publication
(10) Pub. No.: US 2022/0005611 A1
(43) Pub. Date: Jan. 6, 2022

(54) Medical Communication System For Efficiently Displaying Annotation Information Relating To Patients

(71) Applicant: Feedback Medical Limited, London (GB)

(72) Inventors: Thomas James Oakley, London (GB); Michael Peter Hayball, London (GB); Antoine Saillant, London (GB); Stephen James Brown, London (GB)

(73) Assignee: Feedback Medical Limited, London (GB)

(21) Appl. No.: 16/921,178

(22) Filed: Jul. 6, 2020

(51) Int. Cl. G16H 80/00; G16H 40/20; G16H 10/60

(52) U.S. Cl. CPC G16H 80/00; G16H 10/60; G16H 40/20

(57) ABSTRACT

A medical communication system for efficiently displaying annotation information relating to patients; more particularly annotation records for digital medical assets, such as a digital image data object, which define information to be displayed with the digital medical assets. The medical communication system comprises a user store comprising a plurality of user records, a patient store comprising a plurality of patient records, a medical asset store comprising a plurality of digital medical assets, and an annotation store comprising a plurality of annotation records, and is configured to obtain annotation information for a digital medical asset from a first user; generate an annotation record; store the annotation record in the annotation store, associate the annotation record with a patient record associated with the digital medical asset, receive a request from a second user to display the annotation record, and display to the second user the digital medical asset associated with the annotation record together with the annotation information in the annotation record.

### FIELD OF THE INVENTION

The present invention concerns a medical communication system for efficiently displaying annotation information relating to patients. More particularly, but not exclusively, this invention concerns generating and displaying annotation records for digital medical assets, where the annotation records define information to be displayed with the digital medical assets, as well as in the patient record. This innovative combination improves the communication of decision made around such annotations. The invention is particularly advantageous where a digital medical asset is a digital image data object.

### BACKGROUND OF THE INVENTION

It is common for information stored about a patient to include digital medical assets, i.e. files or collections of data, such as text document, videos and images, including collections of related images such as those generated by medical imaging scans. It would be advantageous for medical professionals to be able to share information and queries regarding such digital medical assets. However, known communication systems are not designed to handle such digital medical assets, and so such information cannot be easily shared. It may only be possible, for example, by extracting a part of a digital medical asset, e.g. a part of a single image of a digital medical asset that is a collection of a related image. Such an extracted part may be poor quality, and/or difficult to interpret as the information provided by the rest of the digital medical asset, e.g. the other related images, is not available. The creation of such extracted parts of digital medical assets further consumes additional storage and transmission resources of communication systems because it requires duplicate versions of digital medical assets to be created and communicated. Further, having additional extracted parts of digital medical assets, potentially with amendments made, can create problems with version control. Also, as the extracted parts may need to be stored separately from the digital medical assets (as existing systems to store digital medical assets may not be configured to be able to store them), this can complicate the display of the extracted parts by a graphical user interface.

It would also be advantageous for medical professionals to be able to easily record and retrieve information about patients, for example information about digital medical assets as discussed above, and information about the status of a patient. This includes being able to easily see only patient information that is relevant to them and that they wish to see, and being notified about the existence of updates to patient information.

The present invention seeks to solve and/or mitigate some or all of the above-mentioned problems. Alternatively and/or additionally, the present invention seeks to provide an improved medical communication system for communicating information relating to patients.

### SUMMARY OF THE INVENTION

In accordance with a first aspect of the invention there is provided a medical communication system for efficiently displaying annotation information relating to patients, comprising:

a user store comprising a plurality of user records, wherein each user record is associated with a user of the medical communication system;

a patient store comprising a plurality of patient records, wherein each patient record contains information relating to a patient;

a medical asset store comprising a plurality of digital medical assets, wherein each digital medical asset of the medical asset store is associated with a patient record of the patient store; and

an annotation store comprising a plurality of annotation records, wherein each annotation record is associated with a digital medical asset of the medical asset store;

wherein the medical communication system is arranged to:

obtain from a first user of the medical communication system annotation information for a digital medical asset from the medical asset store;

generate an annotation record comprising the annotation information, the annotation record being distinct from the digital medical asset;

store the annotation record in the annotation store, the annotation record being associated with the digital medical asset;

associate the annotation record with the patient record associated with the digital medical asset;

receive a request from a second user of the medical communication system to display the annotation record; and

display to the second user the digital medical asset associated with the annotation record, or a part thereof, together with the annotation information in the annotation record.

Aspects of the present disclosure offer several technological advancements to medical communication systems. By allowing a first user to create an annotation record defining information to be displayed with a digital medical asset, information relating to the digital medical asset can easily be communicated to a second user. This is because the medical communication system also contains the digital medical assets themselves, and so can display the digital medical asset itself (or part thereof) along with the information provided by the annotation. A relevant part of the digital medical asset does not need to be extracted by the first user to send with the other information, as is the case for known systems. (Or worse, no part of the digital medical asset is sent with the information at all.) In this way, the disadvantage that can result when extracting and sending a relevant part can be avoided, namely that the extraction can be laborious, the extracted part may be of poor quality, and may be difficult to interpret as the rest of the digital medical asset is not easily available. In addition, as the annotation records are created as separate records from the digital medical assets, no change is made to the digital medical asset by the creation of the asset, meaning that the digital medical assets can remain immutable, stored in the medical asset store in their original form. Accordingly, creating and storing the annotation records as separate records reduces the storage and communications resources required by known communications systems that create and store duplicate, and often poorer quality, extracted versions of digital medical assets. In addition, the display of the images and annotations by a graphical user interface is simplified, and can be done more quickly and efficiently, while allowing annotations to be manipulated (e.g. hidden if desired).

The association of the annotation record with the patient record may comprises recording details of the annotation record in patient record.

Advantageously, the patient store comprises: a patient record store comprising the plurality of patient records; and a patient conversation store comprising a plurality of conversation records, wherein each conversation record of the patient conversation store is associated with a patient record of the patient store. In that case, the association of the annotation record with the patient record advantageously comprises recording details of the annotation record in a conversation record in the patient conversation store. In this way, the patient records of the patient store can record the basic information of the patient, e.g. their name, date of birth, hospital number and the like. The conversation records in the patient conversation store can then record messages, notes and the like regarding the patient. Particularly advantageously, as above a conversation record can record detail of an annotation. This provides a link between a digital medical asset (via the annotation record) and a particular part of the patient store, i.e. the conversation record. This allows easy transitioning back and forth between the conversation record in the patient conversation store (and conversation records near to that conversation record, which can be useful to a user for context), and the digital medical asset/part thereof defined by the annotation record.

Advantageously, the digital medical asset of the medical asset store is a medical image data object. The medical image data object may comprise a plurality of images, and the annotation record define that the information to be displayed with the digital medical asset is displayed with an image of the plurality of images selected by the first user. In this way, the first user can easily indicate an image of particular relevance or interest within the medical image data object to be displayed to the second user. However, as the image is not extracted from the medical image data object, the medical communication system can easily allow the second user to view the other images in the medical image data object. Further, it is particularly advantageous for medical image data object to be able to remain immutable and stored in its original form, particular where the format of the medical image data object does not allow for annotations to be added, so to include the annotation in the medical image data object would require the format of the medical image data object to be changed.

The medical image data object may be in DICOM (Digital Imaging and Communications in Medicine) format.

Alternatively, the digital medical asset of the medical asset store may be a document containing information relating to a patient. The document may contain text and/or images, and may be in PDF format, Word format, or any other suitable format. However, it will be appreciated that the digital medical asset could be any other suitable type of file, collection of files or the like.

Advantageously, the annotation record defines a region of interest of the digital medical asset. In this way, the first user can easily indicate a part of a digital medical asset of similar particular relevance or interest. This may for example be a passage of a document, or an area of an image, for example. Where the region of interest is an area of an image, when displayed to the second user the region of interest can be highlighting, for example by having a shape drawn around it or by any other suitable means.

Advantageously, the medical communication system further comprises a label store comprising a plurality of label records, wherein each label record can be selectively associated with one or more patient records. In this case, the medical communication system may be further arranged to: allow a user of the medical communication system to change the association of one or more labels with a patient record; and record in the patient records details of the change of the association of the one or more labels. The change may for example be to associate or disassociate the patient record with a label, or to move the patient record between labels, i.e. to disassociate the patient record with a first label and associate the patient record with a second label in a single step. The association of patient records with labels gives a convenient way to create a workflow to track the status of patient, who has responsibility for them and the like.

Advantageously, each label record in the label store can be dependent upon another label record in the label store, and a user is permitted to associate a patient record with a first label record only if the patient record is already associated with a second label record on which the first label record is dependent. This allows the workflow of a patient record to be controlled, so that a patient record cannot be associated with a label that is not appropriate.

Advantageously, a user record can be associated with one or more patient records.

Advantageously, a user record can be associated with one or more label records, and a user record is associated with a patient record when it is associated with a label with which the patient record is associated. This allows multiple user records to be quickly and reliably associated (and disassociated) with a patient record.

Preferably, a user of the medical message system is only permitted to generate an annotation record for a digital medical asset, when the digital medical asset is associated with a patient record associated with the user record for the user.

Advantageously, the medical communication system is arranged, when an annotation record associated with a digital medical asset is stored in the annotation store, to provide a notification to users whose user records are associated with the patient record with which the digital medical asset is associated. This allows users to be easily notified when there are updates to patients for which they are responsible. A notification may be sent as a message to the user, for example as an email or in “instant message”. Advantageously, the notification may be an indication displayed against the patient records when viewed by the user in the medical communication system.

Preferably, the medical communication system is arranged to display to a user a list of patient records associated with the user record for the user. Preferably, the user is only permitted to see patient records with which their user record is associated. Advantageously, the medical communication system is further arranged to allow the user to select one or more labels with which they are associated, and to filter the list of patient records to display only the patient records associated with the one or more selected labels. Where the medical communication system provides notifications, the list of patient records may include indications that there are notifications.

In accordance with a second aspect of the invention there is provided a computer-implemented method for efficiently displaying annotation information relating to patients using a medical communication system comprising:

a user store comprising a plurality of user records, wherein each user record is associated with a user of the medical communication system;

a patient store comprising a plurality of patient records, wherein each patient record contains information relating to a patient;

a medical asset store comprising a plurality of digital medical assets, wherein each digital medical asset of the medical asset store is associated with a patient record of the patient store; and

an annotation store comprising a plurality of annotation records, wherein each annotation record is associated with a digital medical asset of the medical asset store;

the method comprising the steps of:

obtaining from a first user of the medical communication system annotation information for a digital medical asset from the medical asset store;

generating an annotation record comprising the annotation information, the annotation record being distinct from the digital medical asset;

storing the annotation record in the annotation store, the annotation record being associated with the digital medical asset;

associating the annotation record with the patient record associated with the digital medical asset;

receiving a request from a second user of the medical communication system to display the annotation record; and

displaying to the second user the digital medical asset associated with the annotation record, or a part thereof, together with the annotation information in the annotation record.

In accordance with a third aspect of the invention there is provided a non-transitory computer-readable storage medium comprising instructions for efficiently displaying annotation information which, when executed by a medical communication system comprising:

a user store comprising a plurality of user records, wherein each user record is associated with a user of the medical communication system;

a patient store comprising a plurality of patient records, wherein each patient record contains information relating to a patient;

a medical asset store comprising a plurality of digital medical assets, wherein each digital medical asset of the medical asset store is associated with a patient record of the patient store; and

an annotation store comprising a plurality of annotation records, wherein each annotation record is associated with a digital medical asset of the medical asset store;

cause the medical communication system to carry out the steps of:

obtaining from a first user of the medical communication system annotation information for a digital medical asset from the medical asset store;

generating an annotation record comprising the annotation information, the annotation record being distinct from the digital medical asset;

associating the annotation record in the annotation store, the annotation record being associated with the digital medical asset;

associating the annotation record with the patient record associated with the digital medical asset;

receiving a request from a second user of the medical communication system to display the annotation record; and

displaying to the second user the digital medical asset associated with the annotation record, or a part thereof, together with the annotation information in the annotation record.

It will of course be appreciated that features described in relation to one aspect of the present invention may be incorporated into other aspects of the present invention. For example, the method and computer-readable storage medium of the invention may incorporate any of the features described with reference to the system of the invention and vice versa.

### DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described by way of example only with reference to the accompanying schematic drawings of which:

FIG. 1 is a schematic diagram of a medical communication system in accordance with a first embodiment of the invention.

FIG. 2 is a schematic diagram of a medical image data object used in the medical communication system of FIG. 1.

FIG. 3 shows a patient list web page generated by the medical communication system of FIG. 1.

FIG. 4 is a flowchart showing the operation of the medical communication system to generate the patient list web page of FIG. 3.

FIG. 5 shows a patient record web page generated by the medical communication system of FIG. 1.

FIG. 6 shows options available for the patient record web page of FIG. 5.

FIG. 7 shows workflow options available for the patient record web page of FIG. 5.

FIG. 8 shows a list of labels available to filter the patient list web page of FIG. 3.

FIG. 9 shows a filtered patient list web page generated by the medical communication system of FIG. 1.

FIG. 10 is a flowchart showing the operation of the medical communication system of FIG. 1 to provide notifications for patient records.

FIG. 11 shows another patient record web page generated by the medical communication system of FIG. 1.

FIG. 12 shows a patient record web page generated by the medical communication system of FIG. 1 that includes an annotation.

FIG. 13 shows an image view page showing the annotation of FIG. 12.

FIG. 14 is a flowchart showing the operation of the medical communication system to generate and display an annotation.

FIG. 15 is a flowchart showing the operation of the medical communication system to generate an annotation for a medical image data object containing a plurality of images.

FIG. 16 is a flowchart showing the operation of the medical communication system to display an annotation for a medical image data object containing a plurality of images.

### DETAILED DESCRIPTION

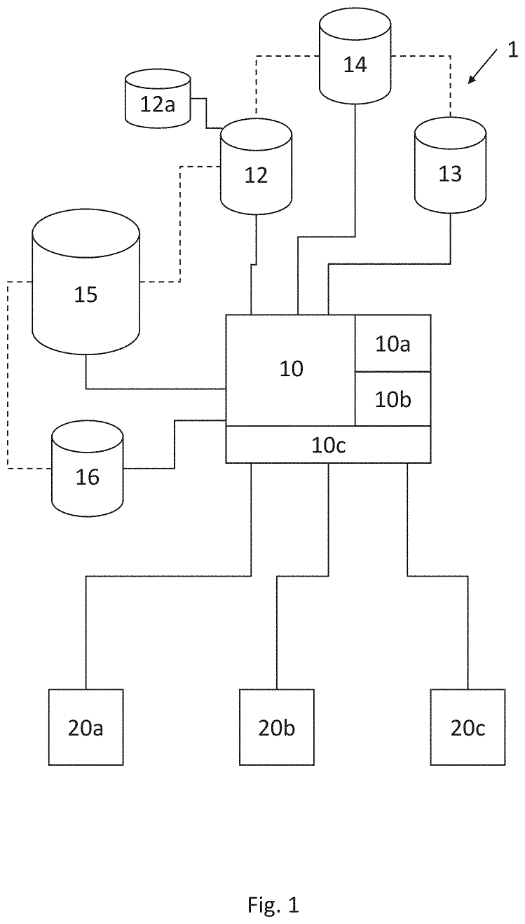

A medical communication system in accordance with an embodiment of the invention is now described, with reference to FIG. 1. The medical communication system 1 comprises a communication server 10, comprising a processor 10a, memory 10b and a web interface 10c.

The communication server 10 also comprises a number of data stores. A first data store is a patient database 12. The patient database 12 comprises a plurality of patient records, each patient record containing details about a patient, such as their name, date of birth, hospital number and the like. Each patient record may also comprise results of medical tests, procedures undergone and the like undergone by the patient. The patient database 12 also comprises a patient conversation database comprising a plurality of conversation records, which record messages, notes and the like regarding a patient, and other information as discussed in detail below.

A second data store is a user database 13 which comprises a plurality of user records, each user record containing details about a user of the medical communication system, for example login details and the like. User records of the user database 13 may also be associated with patient records of the patient database 12, for example if the user has responsibility for the patient.

A third data store is label database 14, which comprises a plurality of labels. Each label is associated with a set of user records from the user database 13. In addition, each label is associated with a set of patient records from the patient database 12. A label may for example correspond to a particular department in a hospital, e.g. the renal department. In that case, the users associated with the label will be the staff of the hospital who are members of the department (or those using the medical communication system 1 who wished to receive notifications for that database, at least). The patients associated with the label will be the patients who are currently under the responsibility of the department.

A label may also reflect the status of a patient with respect to a department, for example there may be a “renal department—admissions” label for patient being newly admitted to the department; a “renal department—admitted” label for patients who have completed admission to the department; a “renal department—discharged” label for patients discharged from the department; and so on.

Another type of label may relate to a particular medical treatment, with the users associated with the label being the hospital staff undertaking the treatment, and the patients associated with the label being those receiving the treatment. It will be appreciated that various other types of labels could be used, for example relating to medical studies or any other appropriate label.

Labels may also be indicated as depending on other labels, the use of which is explained in detail below.

The communication server 10 also comprises a medical asset store 15. The medical asset store 15 comprises a plurality of digital medical assets, as discussed in detail below. Each digital medical asset of the medial asset store 15 is associated with a patient record of the patient database 12.

Finally, the communication server 10 also comprises an annotation database 16. The annotation database 16 comprises a plurality of annotation records, as discussed in detail below. Each annotation record is associated with a digital medical asset of the medial asset store 15.

The medical communication system 1 also comprises a plurality of user devices 20a, 20b and 20c in communication with the communication server 10. The user devices 20a, 20b and 20c may be personal computers (PCs), smartphones, or any other suitable device. The user devices 20a, 20b and 20c communicate with the web interface 10c of the communication server 10, via which the communication server 10 provides webpages to the client devices 20a, 20b and 20c when requested. In this way, a user of a user device, being a user associated with a user record stored in the user database 13, can interact with the medical communication system 1, as discussed in detail below. A user may interact with a user device using a keyboard, mouse, monitor, touchscreen or any other suitable user interface means.

The user devices 20a, 20b and 20c may communicate with the communication server 10 via a local area network (LAN), which may include both wired and wireless (WLAN) communication. Alternatively or additionally, as the user devices 20a, 20b and 20c communicate with the web interface 10c of the communication server 10, they may communicate over the Internet, allowing a user device to be located remotely from the communication server 10.

It will be appreciated that the data used by the medical communication system 1 could be stored and arranged in various different ways in alternative embodiments of the invention. For example, the one or more of the patient database 12, user database 13, label database 14 and medical asset store 15 may be separate from the communication server 10 rather than part of it. In such a case, the communication server 10 may communicate with them over a LAN, the Internet, or by other suitable means. Similarly, one or more of the patient database 12, user database 13, label database 14 and medical asset store 15 may be combined into a single database/store. The information contained in the conversation records of the conversation database may instead be contained directly in the patient records themselves.

As mentioned above, the medical asset store 15 comprises a plurality of digital medical assets. Each digital medical asset is associated with a patient record in the patient database 12.

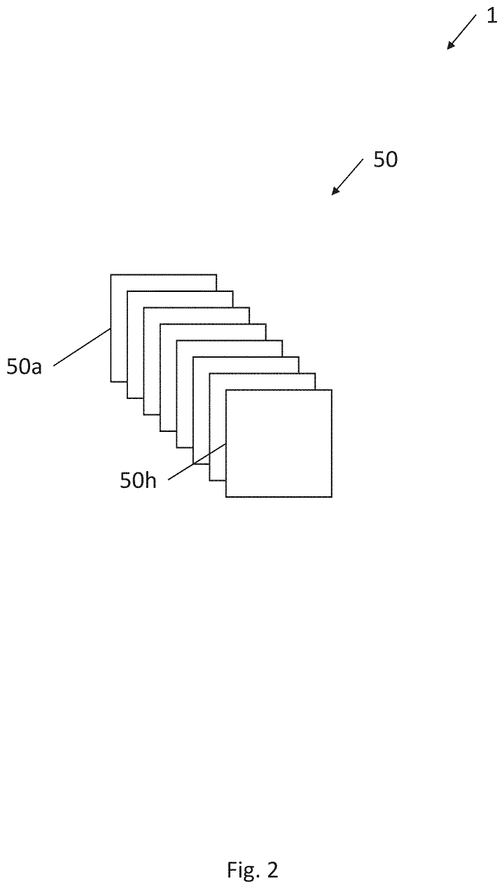

An example of a digital medical asset is a medical image data object. A medical image data object may be a single medical image relating to the patient, for example a chest X-ray of the patient. Alternatively, a medical image data object may be a collection of medical images, as shown by the medical image data object 50 of FIG. 2. It can be seen that the medical image data object 50 comprises a series of images 50a to 50h. Such a medical image data object may be generated by a CT (computed tomography) scan, MRI (magnetic resonance imaging) scan or the like, for example, where a single session of scanning results in a series of images that are “slices” through the scanned part of the patient. Another example of a collection of medical images could be the same part of the body scanned at different time points, such as X-Ray angiograms, or ultrasounds, where the multiple series of images could be displayed as a movie.

A medical image data object may be in the DICOM (Digital Imaging and Communications in Medicine) format. The DICOM format is an International standard framework for communicating digital medical information, particularly medical images.

A DICOM object can contain information relating to a particular patient. The object can be separated into multiple “studies”, where each study corresponds to one group of investigations performed on a patient. For example, all investigations performed on a patient in one location or with one type of equipment (called modality) may be within one study, and investigations performed in a different location (room, etc.) or with different modalities being within a separate study.

Each study can then be separated into multiple series, where each series corresponds a different part of the patient, or using scanning the same body part but with different acquisition parameters, for example. For example, an MRI scan of the patient's head and an MRI scan of the patient's chest may be in different series.

Each series can then contain multiple images, or other appropriate data object(S) obtained by the investigation performed on the patient.

Another example of a digital medical asset is a document containing information about the patient of the patient record with which the digital asset is associated. The document may for example be in PDF format, Word format or any other suitable format. It will be appreciated that a digital medical asset may be any other suitable digital file, collection of files or the like.

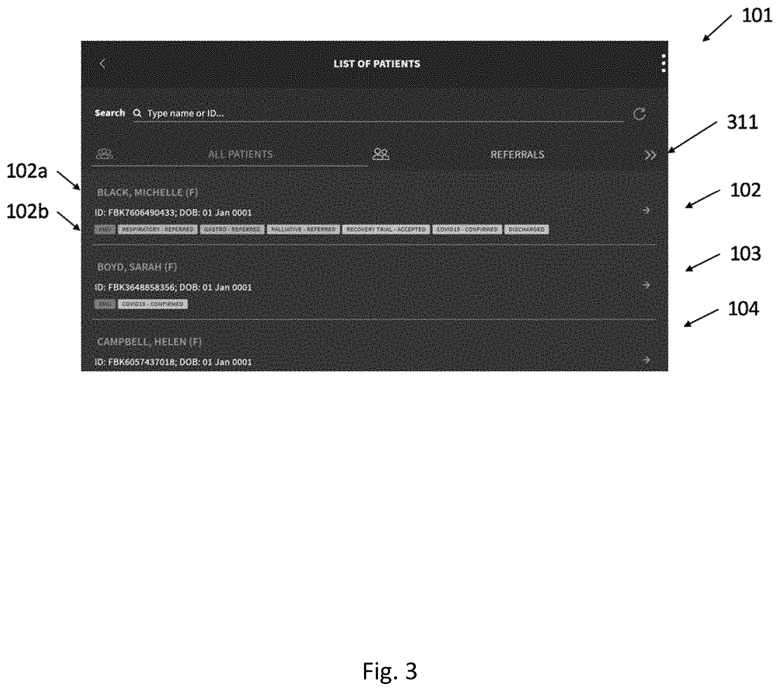

The operation of the medical communication system 1 is now described, with reference to FIGS. 3 to 16. In the following example a user uses the user device 20a of the medical communication system 1.

To connect to the communication server 10, the user uses a web browser on to connect to the web interface 10c of the communication server 10, and request a logon page. The user then logs on using their login credentials, which are checked by the communication server 10 against credentials stored for the user in the user database 13.

When the user has successfully logged on, they are shown the patient list web page shown in FIG. 3. As can be seen, this web page 101 includes a graphical user interface that shows a list of patients 102, 103, 104 which correspond to patient records in the patient database 12. The patients displayed for a particular user are those associated with the user, as described in detail below. Each patient in the list shows certain details relating to the patient, taken from their patient record. For example, the patient 102 has shown their identification details 102a (their name, hospital ID number and date of birth). The patient 102 also has shown the set of labels 102b from the label database 14 with which they are associated.

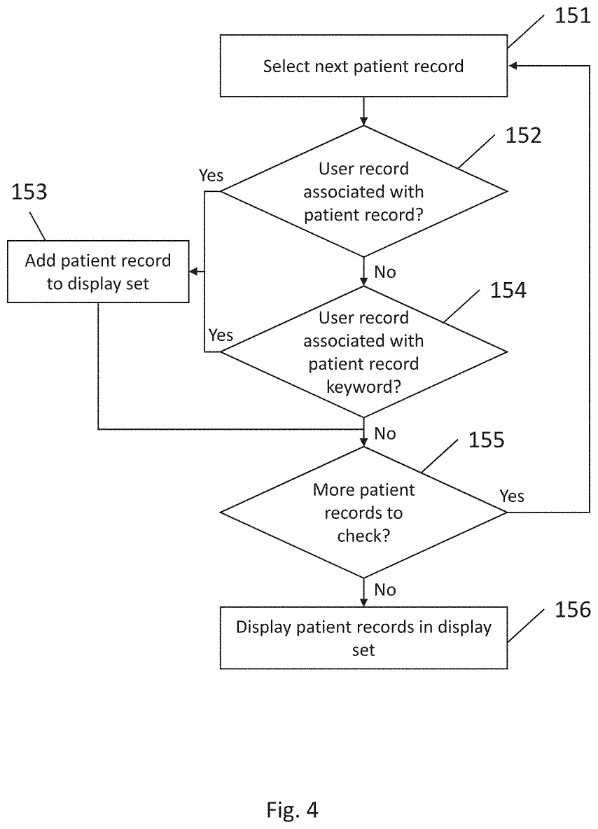

The operation of the communication server 10 to determine the list of patients to display on the patient list web page 101 is shown in the flowchart of FIG. 4. In a first step, a first patient record is selected (step 151). It is then checked if the user record for the logged in user is associated with the patient record (step 152). If so, the patient record is added to a set of patient records to display (step 153). If the user record for the logged in user is not associated with the patient record, it is then checked if the user record is associated with a label that the patient record is also associated with (step 154). Again if so the patient record is added to the set of patient records to display (step 153).

Following a patient record being added to the set of patient records to display (step 153), or following it being determined that a user account is not associated with a label that the patient record is also associated with (step 154), it is the checked if there are more patient records to check (step 155). If there are, the steps are repeated for the next patient record (step 151 again). If not, all patient records have been checked. The set of patient records to display is then those shown by the patient list web page, and it can be seen that it is the set of patient records to which the user is associated, either directly or via a label.

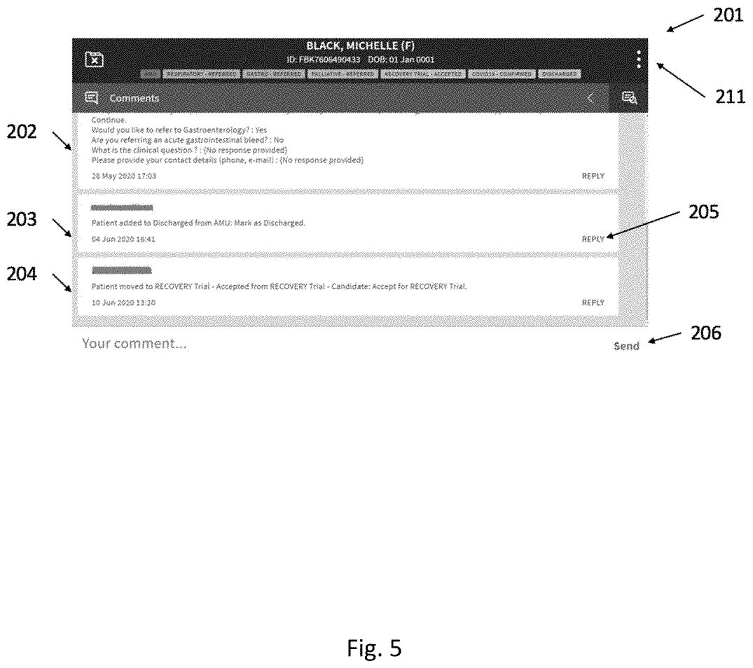

If a patient shown in patient list web page 101 is selected, the patient record web page 201 shown in FIG. 5 is displayed. As can be seen, the patient record web page 201 includes a graphical user interface that shows at the top the patient's identification details and the labels with which they are associated, and then a set of entries 202, 203, 204 of the patient record. Each entry shows information stored in a conversation record, obtained from the patient conversation database 12a by identifying conversation records associated with the patient record. Each entry has a date/time stamp indicating when the conversation record was added to the patient conversation database 12a.

In the example shown in FIG. 5, the first entry 202 shows medical information about the patient, while the second entry 203 and third entry 204 show that a label with which the patient is associated was changed.

At the bottom of the patient record web page 201 is a comment box 206, into which text can be entered so that it is added as a new conversation record in the patient conversation database 12a associated with the patient record. In addition each existing entry has a “reply” option 205, which again allows a new conversation record to be added to the patient conversation database 12a, associated with the existing conversation record to which it is a reply, as well as to the patient record.

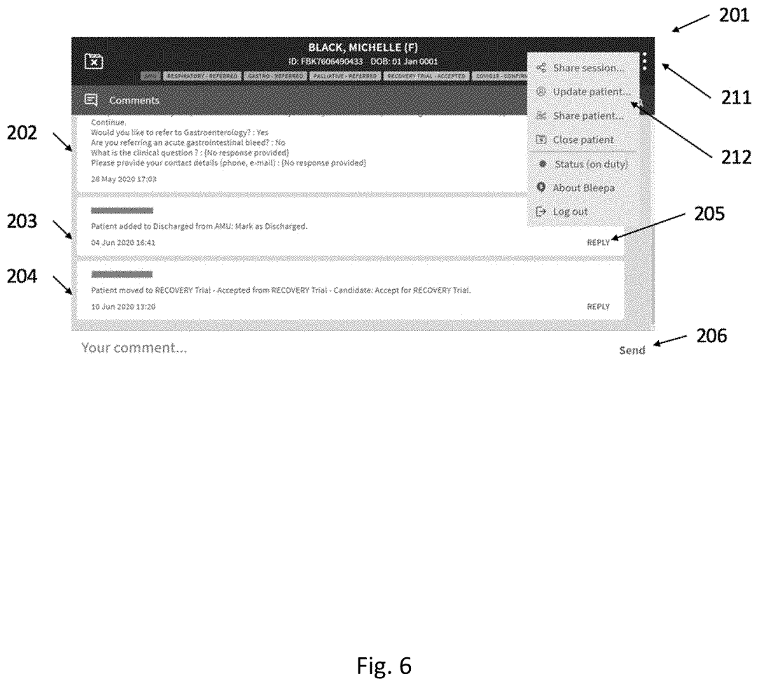

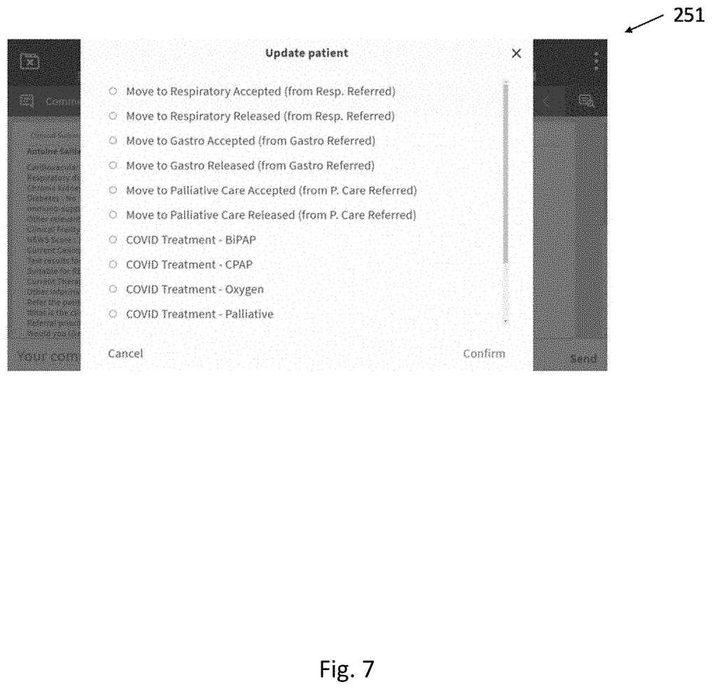

The patent list web page 101 also has in its top right corner a “kebab” icon 211, which displays the set of options shown in FIG. 6. One option is “Update patient” 106. When clicked, a graphical user interface with the workflow options 251 shown in FIG. 7 is displayed. As can be seen, a first set of six options are to move the patient from a first label to a second label. For each, the first label is a label to which the patient is already associated. The second label is then a label which is indicated in the label database 14 to be dependent on the first label. In this way, it can be ensured that a patient is only associated with a label that makes sense; for example, they cannot be associated with the label “renal department—admitted” unless they are currently associated with the label “renal department—admissions”, and they cannot be associated with the label “renal department—discharged” unless they are currently associated with the label “renal department—admitted”.

A second set of four options of the workflow options 251 are then simply to add the patient to a particular label. These labels relate to particular types of condition/treatment and so do not require the patient to currently be part of any existing label. In the label database 14, these labels are not dependent on any other labels.

When an option is selected from workflow options 251, the patient record is associated with the required label (and no longer associated with the required current label, in the case of a move), and the change is also recorded as a conversation record in the patient conversation database 12a, such as shown by the entries 203 and 204.

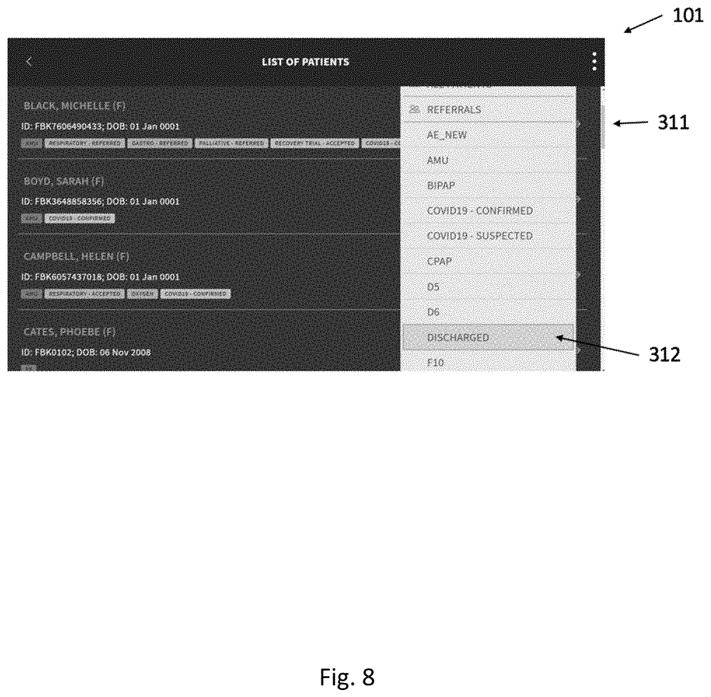

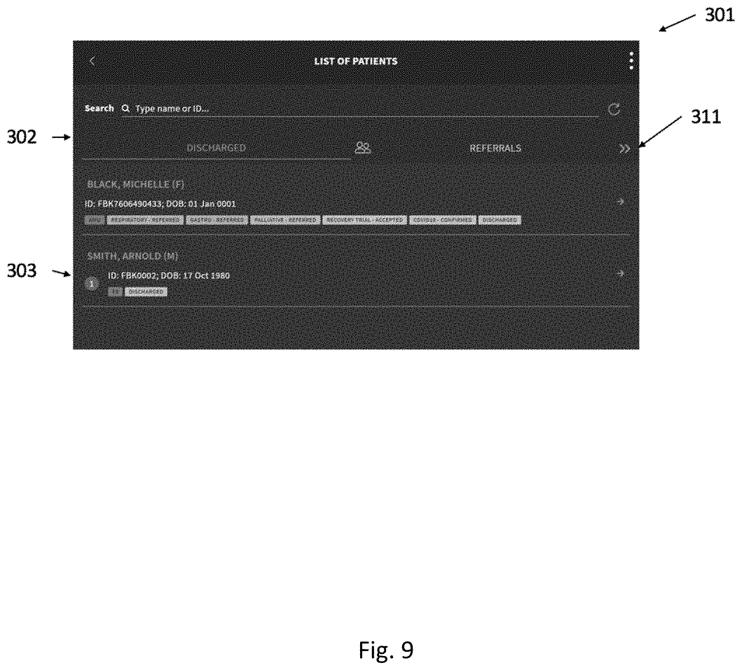

As shown in FIG. 8, a chevrons icon 311 on the patient list web page 101 displays a graphical user interface with a list of the labels with which a patient record can be associated. If the option “DISCHARGED” 312 is selected, then the filtered patient list web page 301 of FIG. 9 is shown. This is similar to the patient list web page 101, but filtered so that only patient records associated with the selected label are shown. In the example of FIG. 9, it can be seen that the filtered patient list web page 301 now has a heading 302 showing that DISCHARGED has been selected, and now only shows two patient records both associated with the DISCHARGED label. The list of patients to display on the filtered patient list web page 301 is determined in a similar way to the list of patients for the patient list web page 101, i.e. using the process of FIG. 4, except that the only patient records considered (i.e. selected in step 151) are those associated with the relevant label, i.e. the DISCHARGED label in the current example.

In addition, it can be seen that the second patient record is showing a notification 303, which is a number 1 within a red circle. This provides a notification to the user that new information has been added to the second patient record since the last time it was viewed by the user.

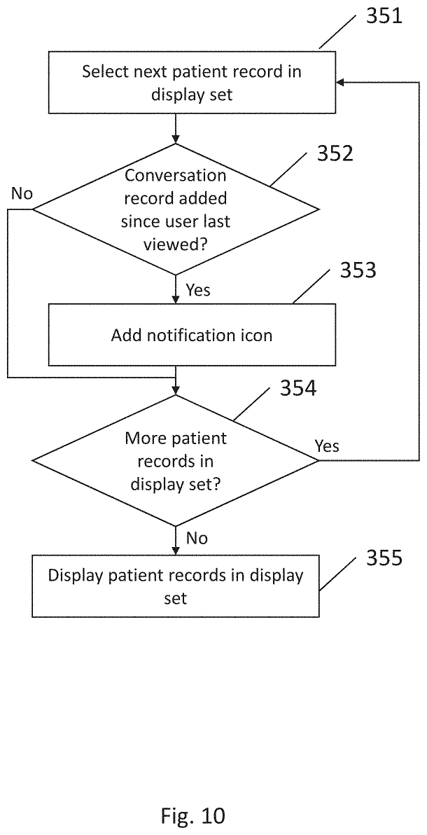

The operation of the communication server 10 to provide the notifications for the patient records is shown in the flowchart of FIG. 10. The communication server 10 uses the list of patient records to display generated as above. In a first step, a first patient record from the list of patient records to display is selected (step 351). It is then checked if a new conversation record associated with the patient record has been added to the patient conversation database 12a, since the user last viewed the patient record (step 352). (It may also be checked if the patient record itself has changed since last viewed by the user.) If so, this indicates there is a conversation record that the user has not seen, and so a notification is added to be displayed with the patient record (step 353). In the present example the notification is an icon showing the number of conversation records that have been added to the patient record since it was last viewed by the user, but it will be appreciated that various other types of notification could be used.

It is then checked if there are any more patient records in the display set to be checked (step 354). If so, the next patient record from the display set is selected (step 351), and the process repeated for the newly selected patient record. If there are no further patient records to check, the set of patient records are displayed (step 355), along with their notifications where appropriate.

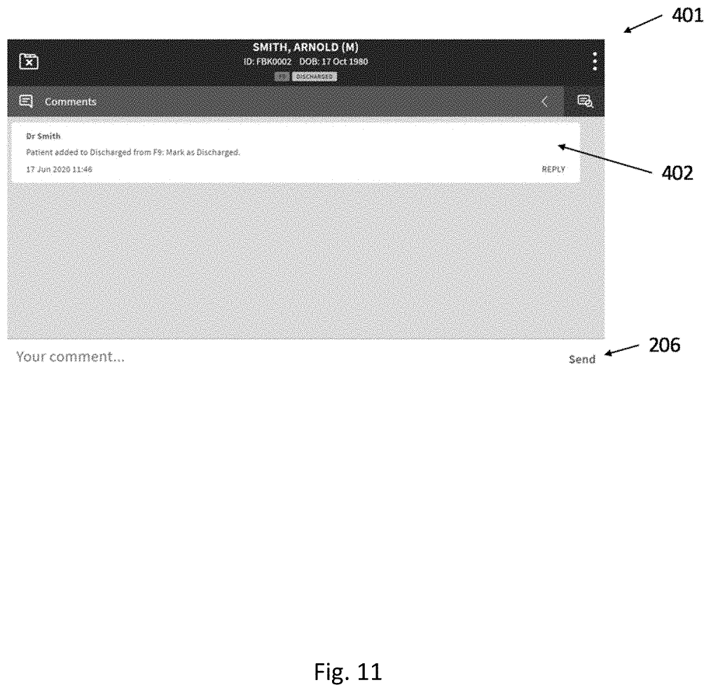

FIG. 11 shows the patient record web page 401 for the patient record of the filtered patient list web page 301 with the notification 302. It can be seen that the new entry 402 was a comment, left by another user using the comment box 206.

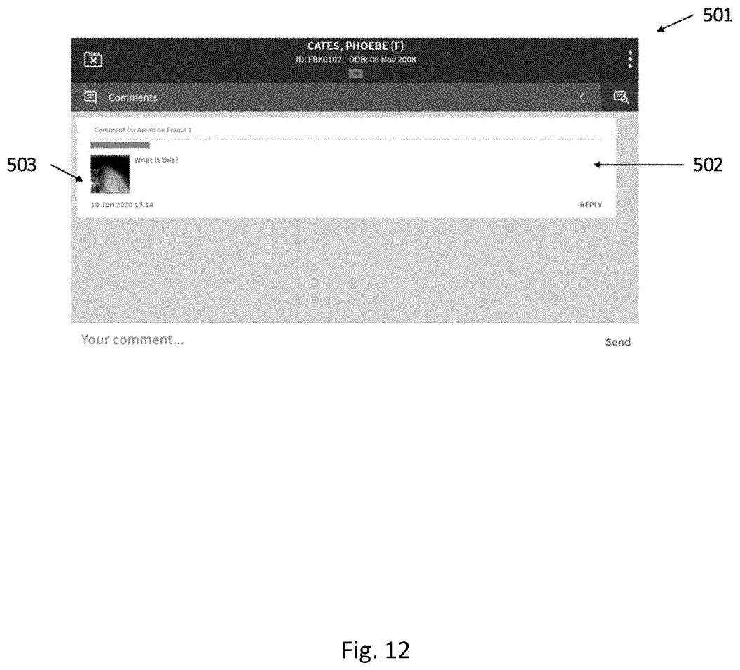

FIG. 12 shows the patient record web page 501 for the patient record of another patient. In this case, the patient record contains an entry 502, which corresponds to a conversation record in the patient conversation database 12a containing details of an annotation record stored in the annotation database 16, associated with a medical image data object from the asset store 15. The entry 502 shown on the patient record web page 501 includes a thumbnail of an image 503 from the medical image data object and some text (“What is this?”).

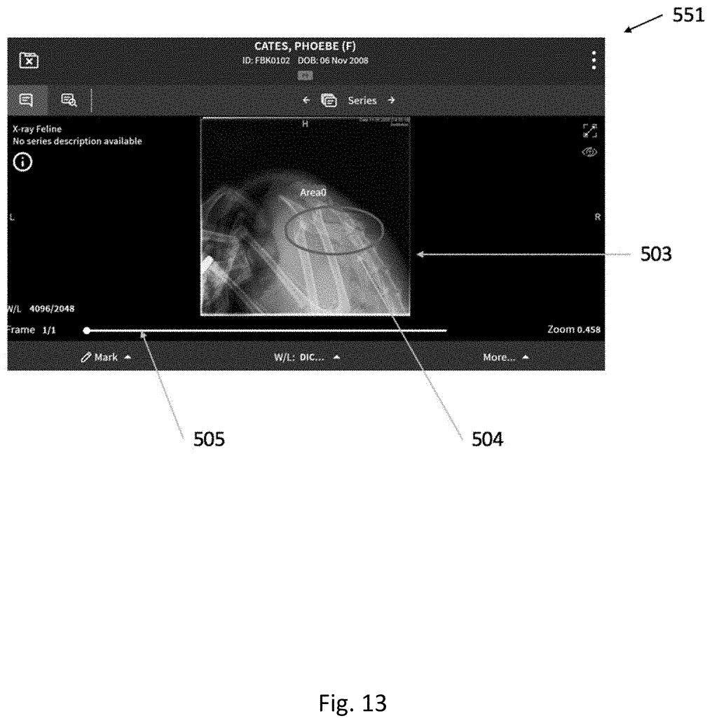

When the image thumbnail is selected, the image view page 551 of FIG. 13 is displayed. The image view page 551 includes a graphical user interface that displays the original version of the image 503, obtained from the medical asset store 15. In addition, the annotation record of the entry 205 is obtained from the annotation database 16. The annotation record is stored in XML format, but it will be appreciated that any suitable format could be used. The XML provides instructions to the graphical user interface as to what to display as an annotation on the original version of the image 503. In particular, the XML instructions have defined that a red oval that indicates a region of interest (ROI) 504 is displayed on the image 503, i.e. a part of the image 503 to which the user who created the entry 502 wished to bring particular attention. The details of the ROI 504 of the image 503 forms the annotation of the medical image data object, stored as the XML annotation record in the annotation database 16. The conversation record in the patient conversation database 12a corresponding to the entry 502 then contain details of the annotation record, and the text “What is this?” of the entry 502. However, it will be appreciated that the information recorded by the entry 502 and the annotation could be stored in other ways, for example the text of text “What is this?” of the entry 502 could be stored in the annotation record rather than directly in the conversation record.

The image view page 551 also contains a slider 505. In the example of FIG. 12, to the left of the slider is an indication “Frame 1/1”, which shows the medical image data object only contains a single image. However, where a medical image data object contained a series of images (or frames), for example 20 images, the indication would be for example “Frame 10/20”, and the slider could be moved to allow the user to easily see the other images in the series. This allows another user viewing the image to easily move to other images in the medical image data object. This can help the user identify an object identified by the ROI 504, or to see the physical context of an object identified by the ROI 504 (e.g. physical structures/conditions in the general area), for example.

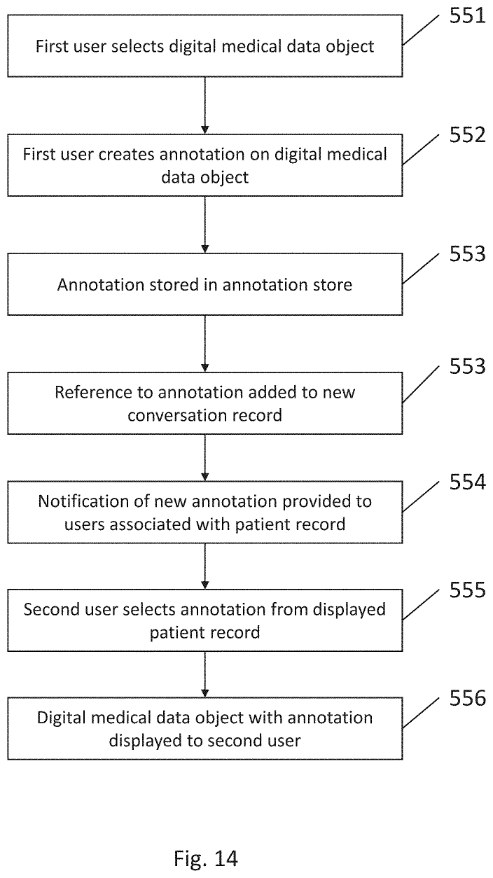

The operation of the communication server 10 to allow a first user to create an annotation of a digital medical asset, and a second user to view the annotation, is shown in the flowchart of FIG. 14. In a first step, the first user selects a digital medical asset from the medical asset store 15 on which they wish to create an annotation (step 551). The user then creates their annotation (step 552). This may be done by selecting an area or areas of the digital medical asset to highlight, adding a text comment for the annotation, adding graphics, or any other desired method. The details of the annotation made by the first user are stored in the annotation database 16 in XML format (step 553).

A reference to the newly created annotation record is then added to a new conversation record in the patient conversation database 12a, associated with the relevant patient record in the patient database 12 (step 553), i.e. the patient record associated with the digital medical asset associated with the annotation. The new conversation record allows the relevant users to be notified of the existence of the new annotation (step 554), as described above.

Next a second user, i.e. one notified in the previous step, views the patient record and selects the entry that references the annotation (step 555). The entry will show some details of the annotation taken from the annotation record, for example as the entry 502 discussed above shows text and a thumbnail of the image. The annotation record is then obtained from the annotation database 16 using the reference to it stored in the conversation record associated with the entry 502, and the medical image data object associated with the annotation record is obtained from the medical asset store 15. The medical image data object (or relevant part) along with the annotation is then displayed to the second user, for example as the image view page 551 discussed above with the ROI 504 being part of the annotation.

An example annotation record in XML format is now described. The XML content of the annotation record is as follows:

<ANNOTATION AnnotTextMode=“Transparency”>

<OBJECT Id=“0” Name=“Area0” Guid=“d98ea2b3-7ebd-

47d9-81b3-9b4e3c527e47”
ImageUID=“1.2.826.0.1.3680043.2.91.5.20190820163931.
51.1”
StudyUID=“1.2.826.0.1.3680043.2.91.5.20190820163931.
50” FrameOfReferenceUID=“”
ImageOrientation=“0\0\0\0\0\0” IsTexRADRegion=“true”
IsFrozen=“true” Type=“ROI” Shape=“polygon”
IsPixelRatioDefault=“True” UserId=“944859fb-6745-
4bb0-bf56-b7f9f0d1906a”>

<PEN>

<COLOR>#ffe119</COLOR>
<THICKNESS>3</THICKNESS>

</PEN>
<GEOM>

<FIGURES>

<FIGURE>

<SEGMENTS>

<SEGMENT Type=“PLine”>

<POINTS>

<P>

<X>708.4662231320367</X>
<Y>1368.8276306405621</Y>

</P>
...
<P>

<X>708.4662231320367</X>
<Y>1368.8276306405621</Y>

</P>

</POINTS>

</SEGMENT>

</SEGMENTS>

</FIGURE>

</FIGURES>

</GEOM>

</OBJECT>

</ANNOTATION>

As can be seen, the XML contains various values identifying the annotation (ID, Name and GUID), and identifying the image to which it applies (ImageUID, StudyUID) and the user who created it (UserID). The XML also contains values indicating the nature of the annotation. These include that the Type of the annotation is ROI (region of interest), its Shape is a polygon. The COLOR and THICKNESS values within the PEN element define the nature of the lines making up the polygon, with the X and Y values in the P elements of the GEOM element giving the positions of the ends of edges (SEGMENTS) of the polygon. In the above example only two P elements are shown, with more indicated by the ellipsis.

It will be appreciated that the XML could define annotations consisting of various other shapes (e.g. rectangles, ovals), or other types of annotation element (e.g. text labels at defined positions in the image). The XML could also define annotations comprising multiple shapes and/or types of annotation element.

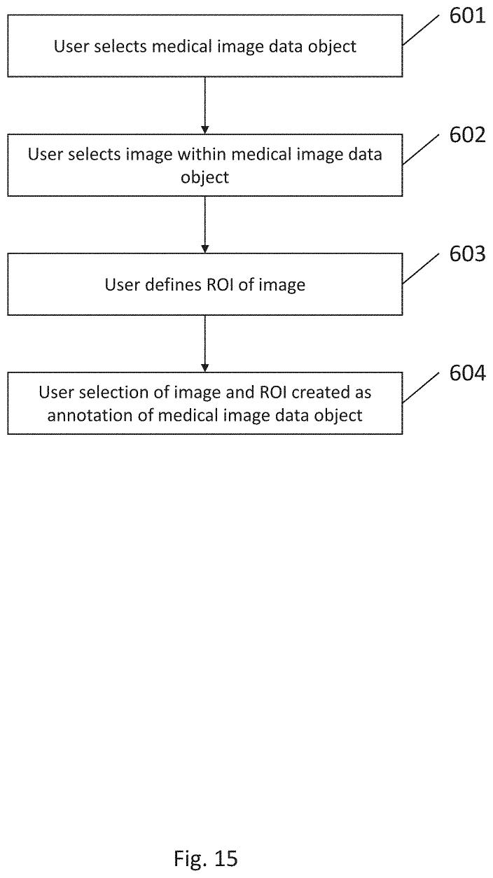

The operation of the communication server 10 to allow a user to create an annotation of a digital medical asset where the digital medical asset is a medical image data object comprising a plurality of images is shown in FIG. 15. In a first step, the user selects a medical image data object (step 601). The user then selects an image from the images of which the medical image data object comprises (step 602), and defines an ROI of the image (step 603). This may be done for example by drawing a circle or oval around the ROI, drawing a freehand line around the ROI, adding an arrow pointing to the ROI, or any other desired means. The details of the image within the medical image data object selected by the user, and the defined ROI, are used by the communication server 10 to create an XML file which is stored in the annotation database 16, as an annotation record associated with the medical image data object.

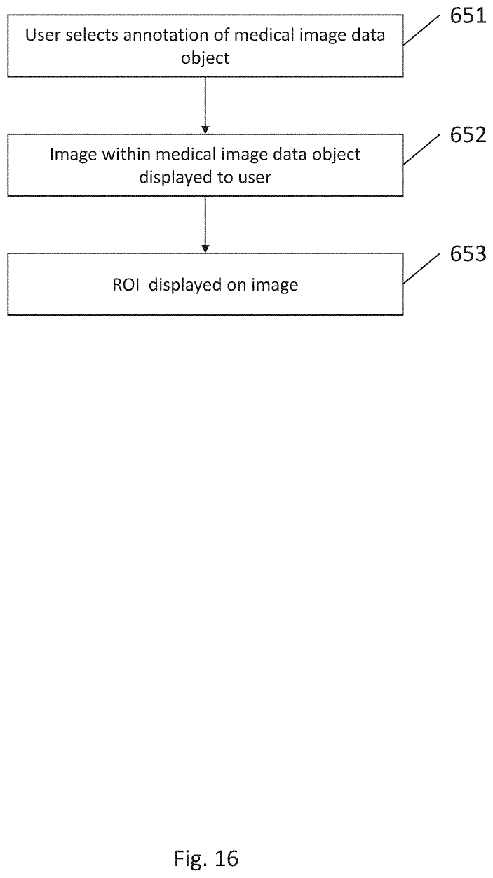

The operation of the communication server 10 to allow a user to view an annotation of a digital medical asset where the digital medical asset is a medical image data object comprising a plurality of images is shown in FIG. 16. In a first step, the user selects the annotation they wish to view (step 651). As discussed above, in practice this will be done by selecting an entry in a patient record that refers to the annotation record for the annotation. The annotation record is an XML file as discussed above, which includes details of a particular image within the medical image data object, and the communication server 10 extracts the details of the particular image from the XML file and displays it to the user (step 652). The XML file will also include details of a defined ROI of the image, and the communication server 10 extracts these details to display the ROI on the image (step 653). Any other appropriate details of the annotation are displayed to the user, on or separate from the image as appropriate. Thus, in this way the user can be displayed an image view page similar to the image view page 551 discussed above, but with the required image from the plurality of images of which the medical image data object comprises being immediately displayed.

While the present invention has been described and illustrated with reference to particular embodiments, it will be appreciated by those of ordinary skill in the art that the invention lends itself to many different variations not specifically illustrated herein. By way of example only, certain possible variations will now be described.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

1. A medical communication system for efficiently displaying annotation information relating to patients, comprising:
a user store comprising a plurality of user records, wherein each user record is associated with a user of the medical communication system;
a patient store comprising a plurality of patient records, wherein each patient record contains information relating to a patient;
a medical asset store comprising a plurality of digital medical assets, wherein each digital medical asset of the medical asset store is associated with a patient record of the patient store; and
an annotation store comprising a plurality of annotation records, wherein each annotation record is associated with a digital medical asset of the medical asset store;
wherein the medical communication system is arranged to:
obtain from a first user of the medical communication system annotation information for a digital medical asset from the medical asset store;
generate an annotation record comprising the annotation information, the annotation record being distinct from the digital medical asset;
store the annotation record in the annotation store, the annotation record being associated with the digital medical asset;
associate the annotation record with the patient record associated with the digital medical asset;
receive a request from a second user of the medical communication system to display the annotation record; and
display to the second user the digital medical asset associated with the annotation record, or a part thereof, together with the annotation information in the annotation record.

2. A medical communication system as claimed in claim 1, wherein the association of the annotation record with the patient record comprises recording details of the annotation record in patient record.

3. A medical communication system as claimed in claim 1, wherein the patient store comprises:
a patient record store comprising the plurality of patient records; and
a patient conversation store comprising a plurality of conversation records, wherein each conversation record of the patient conversation store is associated with a patient record of the patient store.

4. A medical communication system as claimed in claim 3, wherein the association of the annotation record with the patient record comprises recording details of the annotation record in a conversation record in the patient conversation store.

5. A medical communication system as claimed in claim 1, wherein the digital medical asset of the medical asset store is a medical image data object.

6. A medical communication system as claimed in claim 5, wherein the medical image data object comprises a plurality of images, and wherein the annotation record defines that the information to be displayed with the digital medical asset is displayed with an image of the plurality of images selected by the first user.

7. A medical communication system as claimed in claim 1, wherein the digital medical asset of the medical asset store is a document containing information relating to a patient.

8. A medical communication system as claimed in claim 1, wherein the annotation record defines a region of interest of the digital medical asset.

9. A medical communication system as claimed in claim 1, further comprising a label store comprising a plurality of label records, wherein each label record can be associated with one or more patient records.

10. A medical communication system as claimed in claim 9, further arranged to:
allow a user of the medical communication system to change the association of one or more labels with a patient record; and
record in the patient records details of the change of the association of the one or more labels.

11. A medical communication system as claimed in claim 9, wherein each label record in the label store can be dependent upon another label record in the label store, and wherein a user is permitted to associate a patient record with a first label record only if the patient record is already associated with a second label record on which the first label record is dependent.

12. A medical communication system as claimed in claim 1, wherein a user record can be associated with one or more patient records.

13. A medical communication system as claimed in claim 9, wherein a user record can be associated with one or more label records, and wherein a user record is associated with a patient record when it is associated with a label with which the patient record is associated.

14. A medical communication system as claimed in claim 12, wherein a user of the medical message system is only permitted to generate an annotation record for a digital medical asset, when the digital medical asset is associated with a patient record associated with the user record for the user.

15. A medical communication system as claimed in claim 12, arranged, when an annotation record associated with a digital medical asset is stored in the annotation store, to provide a notification to users whose user records are associated with the patent record with which the digital medical asset is associated.

16. A medical communication system as claimed in claim 12, arranged to display to a user a list of patient records associated with the user record for the user.

17. A medical communication system as claimed in claim 16, further arranged to allow the user to select one or more labels with which they are associated, and to filter the list of patient records to display only the patient records associated with the one or more selected labels.

18. A computer-implemented method for efficiently displaying annotation information relating to patients using a medical communication system comprising:
a user store comprising a plurality of user records, wherein each user record is associated with a user of the medical communication system;
a patient store comprising a plurality of patient records, wherein each patient record contains information relating to a patient;
a medical asset store comprising a plurality of digital medical assets, wherein each digital medical asset of the medical asset store is associated with a patient record of the patient store; and
an annotation store comprising a plurality of annotation records, wherein each annotation record is associated with a digital medical asset of the medical asset store;
the method comprising the steps of:
obtaining from a first user of the medical communication system annotation information for a digital medical asset from the medical asset store;
generating an annotation record comprising the annotation information, the annotation record being distinct from the digital medical asset;
storing the annotation record in the annotation store, the annotation record being associated with the digital medical asset;
associating the annotation record with the patient record associated with the digital medical asset;
receiving a request from a second user of the medical communication system to display the annotation record; and
displaying to the second user the digital medical asset associated with the annotation record, or a part thereof, together with the annotation information in the annotation record.

19. A non-transitory computer-readable storage medium comprising instructions for efficiently displaying annotation information which, when executed by a medical communication system comprising:
a user store comprising a plurality of user records, wherein each user record is associated with a user of the medical communication system;
a patient store comprising a plurality of patient records, wherein each patient record contains information relating to a patient;
a medical asset store comprising a plurality of digital medical assets, wherein each digital medical asset of the medical asset store is associated with a patient record of the patient store; and
an annotation store comprising a plurality of annotation records, wherein each annotation record is associated with a digital medical asset of the medical asset store;
cause the medical communication system to carry out the steps of:
obtaining from a first user of the medical communication system annotation information for a digital medical asset from the medical asset store;
generating an annotation record comprising the annotation information, the annotation record being distinct from the digital medical asset;
associating the annotation record in the annotation store, the annotation record being associated with the digital medical asset;
associating the annotation record with the patient record associated with the digital medical asset;
receiving a request from a second user of the medical communication system to display the annotation record; and
displaying to the second user the digital medical asset associated with the annotation record, or a part thereof, together with the annotation information in the annotation record.
